(19) United States
(12) Patent Application Publication
(10) Pub. No.: US 2007/0134705 A1
(43) Pub. Date: Jun. 14, 2007

(54) Method and apparatus for predicting regulation of multiple transcription factors

(75) Inventors: Ho Youl Jung, Daejeon-city (KR); Ji Eun Kim, Busan-city (KR); Seon Hee Park, Daejeon-city (KR)

Correspondence Address: MAYER, BROWN, ROWE & MAW LLP, 1909 K STREET, N.W., WASHINGTON, DC, 20006, US

(73) Assignee: ELECTRONICS AND TELECOMMUNICATIONS RESEARCH INSTITUTE

(21) Appl. No.: 11/634,922

(22) Filed: Dec. 7, 2006

(30) Foreign Application Priority Data
Dec. 8, 2005 (KR) 10-2005-0119278
May 24, 2006 (KR) 10-2006-0046520

(51) Int. Cl. C12Q 1/68; G06F 19/00

(52) U.S. Cl. 435/6; 702/20

(57) ABSTRACT

Provided are a method and apparatus for predicting a regulation of multiple transcription factors which can predict a regulation correlation between the multiple transcription factors and a target gene, wherein the regulation correlation is used in a method of manipulating a gene inside an actual cell. The method includes: separating gene expression profile data into expression profile data of a gene which expresses a transcription factor and an expression profile data of a target gene; clustering all pairs which can be combined, one pair including one transcription factor and one target gene; showing a result of the clustering using an interval graph; and calculating a optimum subset of the transcription factors, which occupies the maximum expression section of the target gene with the minimum number of transcription factors.

### CROSS-REFERENCES TO RELATED PATENT APPLICATION

This application claims the benefit of Korean Patent Application Nos. 10-2005-0119278, filed on Dec. 8, 2005, and 10-2006-0046520, filed on May 24, 2006 in the Korean Intellectual Property Office, the disclosures of which are incorporated herein in their entirety by reference.

### BACKGROUND OF THE INVENTION

1. Field of the Invention

The present invention relates to a method and apparatus for predicting a regulation of multiple transcription factors, and more particularly, to a method and apparatus for predicting a regulation of multiple transcription factors by using gene expression profile data obtained from a micro-array experiment, or the like.

2. Description of the Related Art

In a regulation of transcription factors, at least one transcription factor regulates transcription or expression of at least one gene. The regulation of the transcription factors is an important issue in post genome biology. Since the transcription factors are also generated by transcription or expression of the genes, it can be said that the regulation of the transcription factors can be easily determined by analyzing gene expression data. However, a correlation between the regulation of the transcription factors and expression time transition of the pair of genes is not clear.

The regulation of the transcription factors is important in understanding basic cell functions, such as growth control, cell cycle process, and cell cycle generation, and specialized cell functions, such as hormone secretion and cell to cell communication. At a basic level, the regulation of the transcription factors determines a gene to be transcribed and time for transcribing the gene. Determining a transcription factor which controls expression of a gene can provide an additional insight to expression which is generally wrongly controlled in various human diseases.

Gene profile data obtained through a micro-array experiment shows an aspect of gene expression, and a transcription factor expression gene and a pair of target genes in a regulation correlation show similar aspects of gene expression. Accordingly, when a transcription factor expression gene and a pair of target genes having similar aspects of gene expression are found, it can be predicted that the transcription factor and the target genes are in regulation correlation.

To predict a regulation of transcription factors, various methods of analyzing gene expression patterns have been suggested. Conventionally, the above methods predict that there is a regulation correlation between a transcription factor and a target gene when an expression amount of the transcription factor and an expression amount of the target gene increase together. The conventional methods include a gene clustering method and a Bayesian network method.

The gene clustering method includes grouping data based on similarities between the data and congregating data which has high similarity in the same group and data which has low similarity in different groups. However, the gene clustering method can only predict a regulation correlation when expression patterns are mostly in accordance.

The Bayesian network method takes a long time to calculate and it is impossible to use the Bayesian network to analyze hundreds of transcription factors and thousands of target genes.

Since conventional methods require a regulation correlation between a transcription factor and a gene to be 1:1, it is impossible to analyze a regulation correlation between multiple transcription factors and a target gene which is N:1.

### SUMMARY OF THE INVENTION

The present invention provides a method and apparatus which can predict a regulation correlation between multiple transcription factors and a target gene, wherein the regulation correlation is used in a method of manipulating a gene inside an actual cell.

According to an aspect of the present invention, there is provided a method of predicting regulation of multiple transcription factors, including: separating gene expression profile data into expression profile data of a gene which expresses a transcription factor and an expression profile data of a target gene; clustering all pairs which can be combined, one pair including one transcription factor and one target gene; showing a result of the clustering using an interval graph; and calculating a optimum subset of the transcription factors, which occupies the maximum expression section of the target gene with the minimum number of transcription factors.

The separating of the gene expression profile data may be performed using transcription factor data.

The clustering may be performed through a local clustering, and a pair of regulation genes which are in a simultaneous correlation, a time-delayed correlation, an inverted correlation, and an inverted and time-delayed correlation may be clustered.

The local clustering may include: calculating a degree of a regulation correlation of all gene pairs which can be combined as a numeral value and obtaining corresponding matrices; and selecting a gene pair, which has a threshold value or higher, using a p-value.

The calculating the subset may be performed using Equation 1 below:
min{|S|} and max{(S₀(1)−S₀(0))+(S₁(1)−S₁(0))+ . . . +(Sₘ(1)−Sₘ(0))} <Equation 1>
wherein S denotes a subset of all transcription factors, 1 denotes a predetermined point in time, and m denotes a predetermined transcription factor.

The method may further include inputting the gene expression profile data and transcription factor data before the separating of the gene expression profile data.

The method may further include correcting missing data values and regularizing the gene expression profile data before the separating of the gene expression profile data.

According to another aspect of the present invention, there is provided an apparatus for predicting regulation of multiple transcription factors, including: a data separating unit which separates gene expression profile data into expression profile data of a gene which expresses a transcription factor and an expression profile data of a target gene; a clustering unit which clusters all pairs which can be combined, one pair including one transcription factor and one target gene; an interval graph generating unit which shows a result of the clustering using an interval graph; and an optimizing unit which calculates a optimum subset of the transcription factors, which occupies the maximum expression section of the target gene with the minimum number of transcription factors.

The data separating unit may separate the gene expression profile data using transcription factor data.

The clustering unit may perform a local clustering, and cluster a pair of regulation genes which are in a simultaneous correlation, a time-delayed correlation, an inverted correlation, and an inverted and time-delayed correlation.

The clustering unit may include: a matrix generating unit which calculates a degree of a regulation correlation of all gene pairs which can be combined as a numeral value and obtains corresponding matrices; and a selecting unit which selects a gene pair, which has a threshold value or higher, using a p-value.

The optimizing unit may calculate the subset using Equation 1 below:
min{|S|} and max{(S₀(1)−S₀(0))+(S₁(1)−S₁(0))+ . . . +(Sₘ(1−Sₘ(0))} <Equation 1>
wherein S denotes a subset of all transcription factors, 1 denotes a predetermined point in time, and m denotes a predetermined transcription factor.

The apparatus may further include an inputting unit which receives the gene expression profile data and transcription factor data.

The apparatus may further include a data processing unit which corrects missing data values and regularizes the gene expression profile data before the separating of the gene expression profile data.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating a method of predicting a regulation of multiple transcription factors according to an embodiment of the present invention;

FIG. 2 is an example of an interval graph generated during an internal graph generating process according to an embodiment of the present invention; and

FIG. 3 is a block diagram illustrating an apparatus for predicting a regulation of multiple transcription factors according to an embodiment of the present invention.

### DETAILED DESCRIPTION OF THE INVENTION

Hereinafter, the present invention will be described more fully with reference to the accompanying drawings, in which exemplary embodiments of the invention are shown.

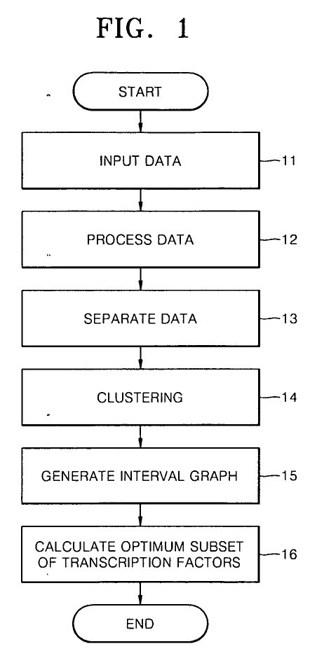

FIG. 1 is a flowchart illustrating a method of predicting a regulation of multiple transcription factors according to an embodiment of the present invention.

Referring to FIG. 1, the method of predicting a regulation of the multiple transcription factors according to the current embodiment of the present invention includes: inputting data (operation 11); processing the data (operation 12); separating the data (operation 13); clustering (operation 14); generating an interval graph (operation 15); and calculating an optimum subset of transcription factors (operation 16).

In operation 11, gene expression profile data and transcription factor data are input.

The gene expression profile data includes expression profile data of a target gene and of a gene expressing a transcription factor. The gene expression profile data may be information regarding a change of an amount of mRNA transcribed from the gene with reference to time. The gene expression profile data may be opened or closed data, and can be obtained through a micro-array experiment, etc.

The transcription factor data is information regarding whether each transcription factor is combined to a promoter region of each target gene. The transcription factor data may be published or unpublished data, and can be directly obtained through a chromatic immunoprecipitation experiment. The chromatic immunoprecipitation experiment is used to determine which base sequence a predetermined protein combines to. Specifically, the chromatic immunoprecipitation experiment can confirm a predetermined promoter region of the genes, which is a region transcription factors regulating gene expression combine, from an entire cell.

In operation 12, the gene expression profile data is converted to a form that can be easily analyzed in a computer. Operation 12 may further include correcting any missing data values and regularizing the gene expression profile data.

The missing data value occurs all the time as a result of the micro-array experiment. As an example, the correcting of the missing data value can be performed by filtering a gene having the missing data values which account for 10% or more of the entire data and using a K-nearest neighbors (KNN) method regarding the remaining genes.

The regularizing of the gene expression profile data involves performing a series of operations to fix an expression average and standard deviation of each gene in a predetermined range. The regularizing can be performed using methods well known to those of ordinary skill in the art. As an example, expression data of each gene can be changed by setting the average to be 0 and the standard deviation to be 1, using a Z-score standardizing method.

In operation 13, the gene expression profile data is separated into expression profile data of a gene expressing a transcription factor and expression profile data of a target gene. The separating of the gene expression profile data may be performed using the transcription factor data, specifically a transcription factor ID.

In operation 14, a regulation correlation of 1:1 is confirmed by clustering all pairs of transcription factors and target genes which can be combined. The clustering divides similar pairs in one group, using the expression profile data of the gene and the expression profile data of the target gene. As a result of clustering, a plurality of clusters may be formed.

The clustering can be performed using various disclosed methods. For example, a hierarchical clustering, a division clustering, and a replication clustering can be used. In hierarchical clustering, clusters have a substructure formed of smaller clusters. The hierarchical clustering is again divided into an agglomerative algorithm in a bottom-up method and a divisive algorithm in a top-down method. The division clustering has no replication and forms the most suitable cluster by repeating a process of allocating each subject to the nearest cluster. For example, the division clustering includes a hard c-means (HCM) algorithm, a k-means algorithm, and ISODATA algorithm. The replication clustering does not have a hierarchical structure between clusters and allows replication of clusters. Accordingly, the most suitable clusters are formed by repeating a process of bringing each subject to the nearest cluster. The replication clustering includes a fuzzy c-means (FCM) algorithm and a b-clump algorithm.

Preferably, the clustering may be performed using a local clustering from expression data of the target gene and expression data of the gene expressing all transcription factors (Jiang Qian et al., J. Mol. Biol. (2001) 314, 1053-1066; Beyond Synexpression Correlations; Local Clustering of Time-shifted and Inverted Gene Expression Profiles Identifies New, Biologically Relevant Interactions).

Using the local clustering, not only a pair of regulation genes in a simultaneous correlation, which can be predicted using a conventional global clustering, can be predicted, but also a pair of regulation genes in a time-delayed correlation, an inverted correlation, or an inverted and time-delayed correlation can be predicted.

A method of the local clustering includes: calculating a degree of a regulation correlation of all gene pairs which can be combined as a numeral value and obtaining corresponding matrices; and selecting a gene pair, which has a threshold value or more, using a p-value. It can be defined that the selected gene pair has 1:1 regulation correlation.

In the gene expression profile data, a measuring point of time 1, 2, 3, . . . , n exists, and xᵢ is an expression level of gene x at a point of time i. Also, matrices of all possible similarity between expression rate of gene x and gene y, such as M(xᵢ,yᵢ)=M_{i,j}=xᵢyⱼ, are considered. For example, two sum matrices E and D are calculated:
E_{i,j}=max(E_{i-1,j-1}+M_{i,j},0)
D_{i,j}=max(D_{i-1,j-1}−M_{i,j},0)

The initial condition of the matrices are E_{0,j}=E_{i,0}=0; D_{0,j}=D_{i,0}=0.

Next, the maximum accumulation score, that is, a local segment having a total sum of M_{i,j}, is obtained using standard dynamic programming as in a local sequence alignment.

Then by comparing the maximum value of the matrices E and D, the overall maximum value S is obtained. S is a match score of two expression profiles. When the maximum values of the corresponding matrices are not diagonal, the two expression profiles are in a time-delayed correlation. The maximum value from the matrix D means that the two expression profiles are in an inverted correlation.

A result of the local clustering can be expressed as follows, against each target gene:
Transcription Factor 1: i, j, k, l
Transcription Factor 2: i′, j′, k′, l′
. . .
Transcription Factor n: i″, j″, k″, I″

Here, i and j are time intervals of a gene which expresses a transcription factor that mostly accords with a target gene, and k and l are time intervals of the corresponding target gene.

In operation 15, the result of the local clustering is shown in an interval graph.

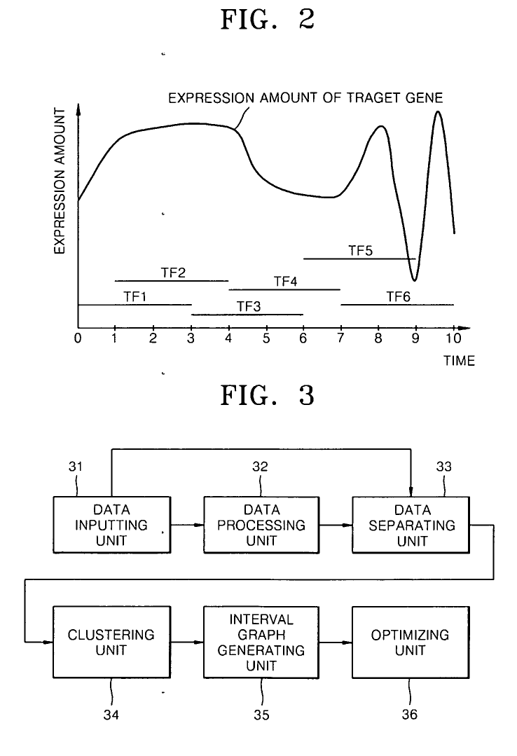

FIG. 2 is an example of an interval graph generated during an internal graph generating process according to an embodiment of the present invention.

Referring to FIG. 2, an expression amount of a target gene is shown, with six transcription factors (TFs), as a result of the local clustering described above, at a predetermined point in time.

Referring back to FIG. 1, in operation 16, a subset of transcription factors, which occupy the maximum expression time period of the target gene with the minimum number of transcription factors, from the transcription factors which are shown in the interval graph of FIG. 2, is calculated.

For example, a subset of transcription factors formed of the transcription factors of periods TF 1, TF 3, and TF 6 shown in FIG. 2 can be multiple transcription factors of the corresponding target gene.

Preferably, a subset of the optimum multiple transcription factors in the interval graph generated after performing the local clustering can be calculated using Equation 1 below:
min{|S|} and max{(S₀(1)−S₀(0))+(S₁(1)−S₁(0))+ . . . +(Sₘ(1)−Sₘ(0))} <Equation 1>

Here, S is a subset of the entire transcription factors.

FIG. 3 is a block diagram illustrating an apparatus for predicting a regulation of multiple transcription factors according to an embodiment of the present invention.

Referring to FIG. 3, the apparatus for predicting a regulation of multiple transcription factors according to the current embodiment of the present invention includes: a data inputting unit 31; a data processing unit 32; a data separating unit 33; a clustering unit 34; an interval graph generating unit 35; and an optimizing unit 36.

The data inputting unit 31 receives gene expression profile data and transcription factor data.

The data processing unit 32 corrects missing data values and regularizes the gene expression profile data.

The data separating unit 33 separates the gene expression profile data into expression profile data of a gene expressing a transcription factor and expression profile data of a target gene. The data separation unit 33 may separate the gene expression profile data using the transcription factor data.

The clustering unit 34 clusters all pairs which can be combined, wherein one pair includes one transcription factor and one target gene.

The clustering unit 34 performs a local clustering and at the same time, clusters a regulation gene pair using a simultaneous correlation, a time-delayed correlation, an inverted correlation, or an inverted and time-delayed correlation.

The clustering unit 34 may include: a matrix generating unit which calculates a degree of a regulation correlation of all gene pairs which can be combined as a numeral value and obtains corresponding matrices; and a selecting unit which selects a gene pair, which has a threshold value or higher, using a p-value.

The interval graph generator 35 shows the result of clustering in the form of an interval graph.

The optimizing unit 36 calculates a subset of the optimum transcription factors, which occupy the maximum expression section of the target gene with the minimum number of the transcription factors, from among the transcription factors.

The optimizing unit may perform the optimization using Equation 1 below:
min{|S|} and max{(S₀(1)−S₀(0))+(S₁(1)−S₁(0))+ . . . +(Sₘ(1)−Sₘ(0))} <Equation 1>

Here, S denotes a subset of all transcription factors, 1 denotes a predetermined point in time, and m denotes a predetermined transcription factor.

The present invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

By providing a method and apparatus which predicts sets of transcription factors that regulate expression of the corresponding genes based on gene expression data, prediction on multiple transcription factors is possible unlike conventional methods and apparatuses. The method is similar to a gene regulation method in an actual cell but can obtain a more accurate result.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of predicting regulation of multiple transcription factors, comprising:
separating gene expression profile data into expression profile data of a gene which expresses a transcription factor and an expression profile data of a target gene;
clustering all pairs which can be combined, one pair including one transcription factor and one target gene;
showing a result of the clustering using an interval graph; and
calculating a optimum subset of the transcription factors, which occupies the maximum expression section of the target gene with the minimum number of transcription factors.

2. The method of claim 1, wherein the separating of the gene expression profile data is performed using transcription factor data.

3. The method of claim 1, wherein the clustering is performed through a local clustering, and a pair of regulation genes which are in a simultaneous correlation, a time-delayed correlation, an inverted correlation, and an inverted and time-delayed correlation are clustered.

4. The method of claim 3, wherein the local clustering comprises:
calculating a degree of a regulation correlation of all gene pairs which can be combined as a numeral value and obtaining corresponding matrices; and
selecting a gene pair, which has a threshold value or higher, using a p-value.

5. The method of claim 1, wherein the calculating the subset is performed using Equation 1 below:
min{|S|} and max{(S₀(1)−S₀(0))+(S₁(1)−S₁(0))+ . . . +(Sₘ(1)−Sₘ(0))} <Equation 1>
wherein S denotes a subset of all transcription factors, 1 denotes a predetermined point in time, and m denotes a predetermined transcription factor.

6. The method of claim 1, further comprising inputting the gene expression profile data and transcription factor data before the separating of the gene expression profile data.

7. The method of claim 1, further comprising correcting missing data values and regularizing the gene expression profile data before the separating of the gene expression profile data.

8. An apparatus for predicting regulation of multiple transcription factors, comprising:
a data separating unit which separates gene expression profile data into expression profile data of a gene which expresses a transcription factor and an expression profile data of a target gene;
a clustering unit which clusters all pairs which can be combined, one pair including one transcription factor and one target gene;
an interval graph generating unit which shows a result of the clustering using an interval graph; and
an optimizing unit which calculates a optimum subset of the transcription factors, which occupies the maximum expression section of the target gene with the minimum number of transcription factors.

9. The apparatus of claim 8, wherein the data separating unit separates the gene expression profile data using transcription factor data.

10. The apparatus of claim 8, wherein the clustering unit performs a local clustering, and clusters a pair of regulation genes which are in a simultaneous correlation, a time-delayed correlation, an inverted correlation, and an inverted and time-delayed correlation.

11. The apparatus of claim 10, wherein the clustering unit comprises:
a matrix generating unit which calculates a degree of a regulation correlation of all gene pairs which can be combined as a numeral value and obtains corresponding matrices; and
a selecting unit which selects a gene pair, which has a threshold value or higher, using a p-value.

12. The apparatus of claim 8, wherein the optimizing unit calculates the subset using Equation 1 below:
min{|S|} and max{(S₀(1)−S₀(0))+(S₁(1)−S₁(0))+ . . . +(Sₘ(1)−Sₘ(0))} <Equation 1>
wherein S denotes a subset of all transcription factors, 1 denotes a predetermined point in time, and m denotes a predetermined transcription factor.

13. The apparatus of claim 8, further comprising an inputting unit which receives the gene expression profile data and transcription factor data.

14. The apparatus of claim 8, further comprising a data processing unit which corrects missing data values and regularizes the gene expression profile data before the separating of the gene expression profile data.
